(19) United States
(12) Patent Application Publication
(10) Pub. No.: US 2007/0004672 A1
(43) Pub. Date: Jan. 4, 2007

(54) Long lasting alginate dry eye, related methods of manufacture and methods of use

(76) Inventors: Dharmendra Jani, Fairport, NY (US); Erning Xia, Penfield, NY (US)

Correspondence Address: Bausch & Lomb Incorporated, One Bausch & Lomb Place, Rochester, NY, 14604-2701, US

(21) Appl. No.: 11/475,277

(22) Filed: Jun. 27, 2006

Related U.S. Application Data
(60) Provisional application No. 60/696,244, filed on Jul. 1, 2005.

(51) Int. Cl. A61K 31/7012; A61K 31/715; A61K 31/05

(52) U.S. Cl. 514/53; 514/54; 514/737

(57) ABSTRACT

The present invention is directed to a composition for treating dry eye comprising, in one embodiment, alginate and a polyol. Such compositions have been found to alleviate the symptoms of dry eye and remain in the eye for a long period of time.

### CROSS REFERENCE

This application claims the benefit of Provisional Patent Application No. 60/696,244 filed Jul. 1, 2005 and is incorporated herein by reference.

### FIELD OF THE INVENTION

This invention relates to a composition for treating dry eye and a related method of use and method of manufacture. In particular, the invention relates to a method of treating dry eye without an active pharmaceutical agent.

### BACKGROUND

Dry eye, also known generically as keratoconjunctivitis sicca and dyslacrima, is a common ophthalmological disorder affecting millions of people. A patient with dry eye may experience burning, a feeling of dryness and persistent irritation. In severe cases, dry eye can seriously impair a person's vision and hence handicap the sufferer in activities such as driving. Certain diseases such as Sjogren's disease manifest dry eye symptoms. Also, as people age, the lacrimal glands in the eye may produce less moisture, resulting in eyes that become dry, inflamed, itchy and gritty.

Although it appears that dry eye may result from a variety of underlying, unrelated pathogenic causes, all presentations of the condition share a common effect, namely the breakdown of the pre-ocular tear film, which commonly results in dehydration of the exposed outer surface and hence the symptoms described above.

A number of approaches exist for the treatment of dry eye. One common approach has been to supplement the ocular tear film using artificial tears instilled throughout the day. Examples of the tear substitute approach include the use of buffered, isotonic saline solutions and aqueous solutions containing water-soluble polymers that render the solutions more viscous and thus less easily shed by the eye by the washing action of the tear fluid. See, for example, U.S. Pat. No. 5,209,927 to Gressel, et al.; U.S. Pat. No. 5,294,607 to Glonek, et al.; and U.S. Pat. No. 4,409,205 to Shively;

Although these approaches have met with some success in some cases, significant challenges in the treatment of dry eye nevertheless remain. Problems include the fact that the use of tear substitutes, while temporarily effective, generally require repeated application over the course of a patient's waking hours, not uncommonly ten to twenty times over the course of a day. Such an approach is not only inconvenient and time consuming, but not very effective in preventing at least the initiation of dry-eye symptoms. Although increasing the viscosity of the dry-eye product may extend the product's duration in the eye or increase in viscosity is effective at extending duration only to a limited extent. Viscous ophthalmic drops are sometimes undesirable because they feel sticky in the eye. Further, increases in the duration of the product would be highly desirable.

Alginate, for the purpose of this application is a polysaccharide that comprises β-D-mannuronic acid and α-L-guluronic acid monomers or salts or derivatives of such acids or salts.

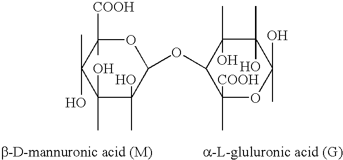

Some alginate polymers are block copolymers with blocks of the guluronic acid (or salt) monomers alternating with blocks of the mannuronic acid (or salt) monomers. Some alginate molecules have single monomers of guluronic acid (or salt) alternating with the comonomers of mannuronic acid (or salt). The ratio and distribution of the M and G components along with the average molecular weight affect the physical and chemical properties of the copolymer. See Haug, A. et al., Acta Chem Scand 20:183-190 (1966). Alginate polymers have viscoelastic rheological properties and other properties that make it suitable for some medical applications. See Klock, G. et al., Biocompatibility of manurononic acid-rich alginates, Biomaterials 18(10): 707-713 (1997).

The use of alginate as a thickener for topical ophthalmic use is disclosed in U.S. Pat. No.6,528,465 and U.S. Publication 2003-0232089 incorporated herein by reference in their entirety. In U.S. Pat. No. 5,776,445, alginate is used as a drug delivery agent that is topically applied to the eye. Particularly, the amount of guluronic acid in the alginate was taught to exceed 50%.

U.S. Patent Publication No.2003/0232089 teaches a dry-eye formulation that contains two polymer ingredients including alginate.

Polyols including glycerin are known as demulcents and tonicity adjusting agents in ophthalmic formulations including formulations for the delivery of a active pharmaceutical agent. See U.S. Pat. Nos. 5,075,104 and 5,209,927 which teach the use of a polyol with a cabomer polymer.

In view of the above, it would be desirable to provide an eye-drop solution that will better alleviate the symptoms of dry eye and that is safe, convenient and economical to use. In particular, it would be highly desirable to develop a product having significantly greater duration of efficacy, in order to significantly decrease the number of times that the product may need to be administered to the eye, over the course of a day, in order to effectively treat the symptoms of dry eye. The present invention addresses these and other needs.

### SUMMARY OF THE INVENTION

The present invention is a dry eye composition comprising an aqueous solution of alginate and a polyol. In one embodiment, the composition does not contain a active pharmaceutical agent. The components of the dry eye composition have improved coating properties and remain in the eye for a longer period of time. They relieve the symptoms of dry eye. The addition of the polyol to the alginate expands the polymer chain dimensions and is believed to result in the enhanced chain dimensions and enhanced binding to the ocular surface.

In another embodiment, the polyol has 2 to 4 carbon atoms, and is preferably glycerin. The concentration of polyol, including glycerin, is a minimum of about 0.01 wt. % and a maximum of about 5 wt. %. The concentration of alginate is a minimum of about 0.01 wt. % and a maximum of about 5 wt. %. In one embodiment, the average molecular weight of alginate is a minimum of about 50 kDa and a maximum of about 5000 kDa.

The present invention also comprises, in one aspect, a method of treating dry eye. The method comprises administering to an eye a composition according to one or more embodiments of the present invention including a composition comprising an aqueous solution of alginate and a polyol to the eye.

In still another embodiment, there is a method for manufacturing a dry eye composition. The method comprises combining, in an aqueous solution, ophthalmically pure alginate with ophthalmically pure polyol. Typically, the ratio of the weight of alginate to weight of polyol in the composition is a minimum of 1:20 and a maximum of 20:1.

The present invention is a dry eye composition that comprises an aqueous solution of alginate having a molecular weight that is a minimum of about 50 kDa and a maximum of about 5000 kDa and a polyol wherein the composition does not contain a active pharmaceutical agent.

In another embodiment, there is a method of manufacturing a dry eye composition comprising combining in an aqueous solution ophthalmically pure alginate having a molecular weight that is a minimum of about 50 kDa and a maximum of about 5000 kDa with ophthalmically pure polyol.

### DETAILED DESCRIPTION OF THE INVENTION

The present invention is directed to a dry eye composition comprising an aqueous solution of alginate and a polyol wherein, in one embodiment, the composition does not contain a active pharmaceutical agent. The composition has been shown to moisturize the eye for a relatively long duration.

The polyol of the present invention is typically a polyol containing 2 to 6 carbon atoms. Preferably, the polyol contains 2 to 4 carbon atoms. The polyol of one embodiment is selected from the group consisting of glycerin, ethylene glycol, poly(ethylene glycol), propylene glycol, sorbitol, manitol and monosaccarides, disaccharides, oligosaccharides and neutral polysaccharide. In one preferred embodiment, the polyol is selected from the group consisting of glycerin, ethylene glycol, propylene glycol, sorbitol, mannitol and monosaccharides. In another preferred embodiment, the polyol is selected from the group comprising disaccharides, oligosaccharides and poly(ethylene glycol). In one preferred embodiment, the polyol is glycerin.

The concentration of polyol including glycerin is a minimum of about 0.01 wt. % about 0.05 wt. % about 0.1 wt. % or about 0.5 wt. %, about 1.0 wt. %, and/or a maximum of about 1.5 wt. %, about 2.0 wt. %, about 3.0 wt. %, about 4.0 wt. % or about 5 wt. % based upon the total weight of the composition.

In one embodiment the polyol is a combination of glycerin and propylene glycol. Typically, the ratio of glycerin to propylene glycol is a minimum of about 30:70, about 35:65, about 40:60, about 45:55. The ratio of glycerin to propylene glycol is a maximum of about 70:30, about 65:35, about 60:40, about 55:45. In one embodiment, the ratio of glycerin to propylene glycol is 1:1. In one embodiment the concentration of glycerin is a minimum of about 0.1 wt. % about 0.3 wt. % about 0.4 wt. % or about 0.5 wt. % and/or a maximum of about 0.8 wt. %, 0.9 wt. %, about 1 wt. %, about 1.5 wt. %, about 2 wt. % or about 3 wt. % based upon the total weight of the composition. In one embodiment the concentration of propylene glycol is a minimum of about 0.1 wt. % about 0.3 wt. % about 0.4 wt. % or about 0.5 wt. % and/or a maximum of about 0.8 wt. %, 0.9 wt. %, about 1 wt. %, about 1.5 wt. %, about 2 wt. % or about 3 wt. % based upon the total weight of the composition.

The present invention includes alginate. Alginate is a polysachharide polymer that has a base unit that is represented by the following formulae:

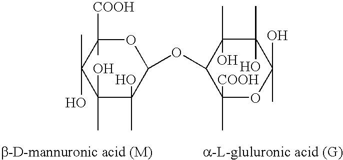

The alginate of one embodiment has a molecular weight that is a minimum of about 50 kDa, about 80 kDa, about 100 kDa, about 500 kDa and/or a maximum of about 5000 kDa, about 2000 kDa, about 1000 kDa, about 700 kDa, about 500 kDa, about 200 kDa, about 100 kDa with ophthalmically pure polyol. In one preferred embodiment, the molecular weight is about 325 kDa.

The alginate of one embodiment, has a ratio of guluronic acid monomer units to mannuronic acid monomer units that is a minimum of about 25:75, about 30:70, about 35:65, or about 40:60. The alginate of an embodiment, has a ratio of guluronic acid monomer units to mannuronic acid monomer units that is a maximum of less than 50:50, about 49:51, about 48:52, about 47:53 or about 46:54. In one embodiment, the ratio of guluronic acid monomer units to mannuronic acid units is about 45:55.

The concentration of alginate is a minimum of about 0.01 wt. % and a maximum of about 2.0 wt. % based upon the total weight of the solution. Typically, the concentration of alginate is a minimum of about 0.05 wt. %, about 0.1 wt. %, about 0.25%, about 0.5 wt. % or about 1 wt. % based upon the total weight of the solution. Typically, the concentration of alginate is a maximum is about 5 wt. %, about 3 wt. %, about 2 wt. %, about 1.5 wt. % and about 1.2 wt. % based upon the total weight of the solution. Preferably, the concentration of alginate is about 0.5 wt. % based upon the total weight of the solution.

In another embodiment, the alginate containing composition is characterized in that it has a Mark-Houwink number that is a minimum of about 0.6. Typically, the Mark-Houwink number is a minimum of about 0.6 and a maximum of about 1.2. In one embodiment, the Mark-Houwink number is about 1.0.

According to one embodiment, the ratio of alginate to polyol is a minimum of about 1:20, about 1:4, about 1:3, about 1:2, about 2:3 or about 3:4 and/or a maximum of about 20:1. about 4:1, about 3:1, about 2:1, about 3:2 or about 4:3.

The present composition may also contain a disinfecting amount or a preservative of an antimicrobial agent. Antimicrobial agents are defined as organic chemicals that derive their antimicrobial activity through a chemical or physiochemical interaction with the microbial organisms. These include sorbic acid, quarternary ammonium polymers and low and high molecular weight biguanides. For example, biguanides include the free bases or salts of alexidine, chlorhexidine, hexamethylene biguanides and their polymers, and combinations of the foregoing. The salts of alexidine and chlorhexidine can be either organic or inorganic and are typically gluconates, nitrates, acetates, phosphates, sulfates, halides and the like. A preferred polymeric biguanide is poly(hexamethylene biguanide) commercially available from Zeneca, Wilmington, Del. under the trademark Cosmocil™ CQ. Generally, the hexamethylene biguanide polymers, also referred to as poly(aminopropyl biguanide) (PAPB), have molecular weights of up to about 100 kDa. A particularly preferred preservative is alexidine.

If used in the subject solution, the antimicrobial agent should be used in an amount which will preserve or prevent the growth of the microorganism population in the formulations employed. Preferably, a preservative amount is that which will reduce the bacterial bioburden after 28 days each by 3 logs and prevents the growth of fungal bioburden by ±0.5 log. Typically, such agents are present in a minimum concentration of about 0.0001 wt. %, 0.0003 wt. % or 0.0005 wt. % and a maximum concentration of about 0.0005 wt. % or 0.001 wt. % or about 0.005 wt. % based upon the total weight of the composition.

The aqueous solutions employed in this invention may contain additional ingredients described above, one or more other components that are commonly present in ophthalmic solutions, for example, buffers, stabilizers, tonicity agents and the like, which aid in making ophthalmic compositions more comfortable to the user. The aqueous solutions of the present invention are typically adjusted with tonicity agents to approximate the tonicity of normal lacrimal fluids which is equivalent to a 0.9 wt. % solution of sodium chloride or a 2.8 wt. % of glycerol solution. The solutions are made substantially isotonic with physiological saline used alone or in combination; otherwise, if simply blended with sterile water and made hypotonic or made hypertonic, the lenses will lose their desirable optical parameters. Correspondingly, excess salt or other tonicity agents may result in the formation of a hypertonic solution that will cause stinging and eye irritation. An osmolality is a minimum of about 200 mOsm/kg, about 225 mOsm/kg, about 250 mOsm/kg, about 260 mOsm/kg, about 280 mOsm/kg, about 300 mOsm/kg or about 320 mOsm/kg and/or a maximum of about 400 mOsm/kg, about 380 mOsm/kg, about 360 mOsm/kg, about 340 mOsm/kg or about 320 mOsm/kg. Most preferably, the osmolality is about 240 mOsm/kg to about 320 mOsm/kg.

Preferably, the composition of at least one embodiment of the present invention has a low ionic strength. Typically, the composition contains low concentration of mono or divalent cations typically found in tear fluids. Generally, the composition contains a low concentration of one or more of the following cations: Na+, K+, Ca++, Mg++, and Zn++. In one embodiment, the concentration of the mono or divalent cations that are typically found in tear fluids (i.e. Na+, K+, Ca++, Mg++ and Zn++) has a minimum concentration of about 0.001 wt. %, about 0.005 wt. %, about 0.01 wt. % or about 0.1 wt. % and/or a maximum of about 0.1 wt. %, about 0.01 wt. %, about 0.1 wt. %, about 0.05 wt. % or about 0.01 wt. % based upon the total weight of the composition.

The pH of the present solutions used to treat dry eye should be maintained at a minimum of about 4 about 5, about 5.5, about 6, about 6.5 and/or a maximum of about 7.5, about 7.8, about 8, about 8.5. Suitable buffers may be added, such as borate, citrate, bicarbonate, aminoalcohol buffers, MOPS buffer, bicine, tricine, TRIS, BIS/TRIS and various mixed phosphate buffers (including combinations of Na₂HPO₄, NaH₂PO₄ and KH₂PO₄) and mixtures thereof. Borate buffers are preferred, particularly for enhancing the efficacy of PAPB. Preferred combination buffers include borate/phosphate and borate/citrate combination buffers. Generally, buffers will be used in amounts having a minimum of about 0.05 wt. % or about 0.1 wt. % and/or a maximum of about 1.5 wt. % or about 2.5 wt. %.

In addition to buffering agents, in some instances it may be desirable to include sequestering agents in the present solutions in order to bind metal ions, which might otherwise react with the lens and/or protein deposits and collect on the lens. Ethylene-diaminetetraacetic acid (EDTA) and its salts (disodium) are preferred examples. They are usually added in amounts having a minimum of about 0.01 wt. % and/or a maximum of about 0.2 wt. %.

The present invention includes a method of treating dry eye comprising administering to an eye a composition comprising an aqueous solution of alginate and a polyol to the eye. In one embodiment the composition does not have a active pharmaceutical agent. The method further includes administering to an eye a composition to any one or more embodiments or combination of embodiments disclosed herein.

In one embodiment, there is a method of manufacturing a dry eye composition. The method of manufacturing comprises combining in an aqueous solution ophthalmically pure alginate (eg. sodium alginate) without adding an active pharmaceutical agent. The alginate of one embodiment has a molecular weight that is a minimum of about 50 kDa, about 80 kDa, about 100 kDa, about 500 kDa and/or a maximum of about 5000 kDa, about 2000 kDa, about 1000 kDa, about 700 kDa, about 500 kDa, about 200 kDa, about 100 kDa with ophthalmically pure polyol. In one preferred embodiment, the molecular weight is about 325 kDa.

The alginate of one embodiment, has a ratio of guluronic acid monomer units to mannuronic acid monomer units that is a minimum of about 25:75, about 30:70, about 35:65, or about 40:60. The alginate of an embodiment, has a ratio of guluronic acid monomer units to mannuronic acid monomer units that is a maximum of less than 50:50, about 49:51, about 48:52, about 47:53 or about 46:54. In one embodiment, the ratio of guluronic acid monomer units to mannuronic acid units is about 45:55.

As indicated above, the present invention is useful for treating dry eye, or, more specifically, its symptoms. For that purpose, compositions for use in the present invention may be sold in a wide range of small-volume containers from 1 ml to 30 ml in size. Such containers can be made from HDPE (high density polyethylene), LDPE (low density polyethylene), polypropylene, poly(ethylene terepthalate) and the like. Flexible bottles having conventional eye-drop dispensing tops are especially suitable for use with the present invention.

The above-described solutions, in accordance with the present invention, may be used by instilling, for example, about one (1) or three (3) drops in the affected eye(s) as needed, for the temporary relief of burning and irritation due to dryness in the eye and for use as a protectant against further irritation, or to relieve dryness to the eye.

### EXAMPLE 1

### Formulation

The following ingredients and respective amounts are used to make Formula 1 of the present invention:

| Formula 1: Ingredient | Mg/gm | | % w/w | |
|---|---|---|---|---|
| Boric Acid | 5.0000 | | 0.5000 | |
| Sodium Borate | 0.1400 | | 0.0140 | |
| Glycerin | 6.0000 | | 0.6000 | |
| Propylene Glycol | 6.0000 | | 0.6000 | |
| Alginate (Medium | 2.5000 | | 0.2500 | |
| Viscosity) | | | | |
| HAP (30%) | 0.5000 | | 0.0500 | |
| Alexidine 2HCl | 3.00 | ppm | 3.00 | ppm |
| Purified Water | Q.S. to 1000.0 | mg | Q.S. to 100% | w/w |

A volume of purified water that is equivalent to from about 85% to about 90% of the total batch weight (the temperature of purified water should be below 40° C. before add any raw material) is added into an appropriate stainless steel mixing vessel. Preferably, the temperature of the purified water should be below 40° C. during this step. Alginate is selected to have a ratio of guluronic monomer units to mannuronic monomer units that is about 45:55. Furthermore, the alginate preferably has an average molecular weight of about 325 kDa. Alginate is added slowly with continued agitation and mixed thereafter for at least 45 minutes.

After the addition of Alginate and corresponding mixing, the following ingredients are slowly added in the order listed and mixed for at least 30 minutes:
Boric Acid
Sodium Borate
HAP (30%)
Glycerin
Propylene Glycol

After these ingredients are mixed, Alexidine HCl was added via a 0.22 μm sterilizing filter and mixed for an additional 30 minutes or more. The preparation is ready for packaging, use and storage. Refrigeration is not needed.

### EXAMPLE 2

### General Testing and Properties

Testing of the Alginate formula was conducted according to standard procedures known-in the art. Specifically, pH, osmolality, clarity, color and viscosity were evaluated for Formula 1 and were reported as follows:

| Test | Property or Characteristic |
|---|---|
| pH @ 25° C. | 6.7-7.1 |
| Osmolality | 220 mOsm/Kg |
| Clarity (visual) | Clear |
| Color | Colorless to pale yellow |
| Viscosity | 7-9 cps |
| Mark-Houwink Number | 1.023 |

### EXAMPLE 3

### Mark-Houwink Number Analysis

Formula 1 was prepared according to Example 1. Formula 1 was analyzed using size exclusion chromatography (SEC) with triple detection. Particularly, lights scattering detection, viscometry trace detection and refractive index detection analysis were performed. The Mark-Houwink number (a) is calculated using the data from the triple detection SEC using the mathematical technique disclosed in Introduction to Physical Polymer Science, Third Edition, L. H. Sperling, Wiley-Interscience, A John Wiley & Sons, Inc., Publication, New York, 2001. Interpretation of the Mark-Houwink number is done according to the following Table 1:

TABLE 1
Values of the Mark-Houwink numbers (a)

| Mark-Houwink numbers (a) | Interpretation |
|---|---|
| 0 | Spheres |
| 0.5-0.8 | Random coils |
| 1.0 | Stiff coils |
| 2.0 | Rods |

A Mark-Houwink number of zero is indicative of a spherical structure. A Mark-Houwink number between 0.5 and 0.8 indicates a physical configuration described as random coils. A Mark-Houwink number above 0.8 indicates a structure that is more ordered than random approaching a stiff coil. A Mark-Houwink number of about 1.0 is a stiff coil and a Mark-Houwink number of 2.0 represents a rod-like structure.

Formula 1 representing Alginate with glycerin and propylene glycol carboxymethylcellulose with no glycerin had a Mark-Houwink number of 1.023 reflecting a stiff coil structure. The stiff coil structure is expected to remain in the eye longer than random coil polymers 0.561 as recorded in Table 2. Thus, without glycerin, carboxymethylcellulose formed a random coil.

While the invention has been described in conjunction with the detailed description and specific examples, this is illustrative only. Accordingly, many alternatives, modifications and variations will be apparent to those skilled in the art in light of the foregoing description and it is, therefore, intended to embrace all such alternatives, modifications and variations as to fall within the spirit and scope of the appended claims.

What is claimed is:

1. A dry eye composition comprising an aqueous solution of alginate and a polyol wherein the composition does not contain a active pharmaceutical agent.

2. The dry eye composition of claim 1, wherein the polyol contains 2 to 6 carbon atoms.

3. The dry eye composition of claim 1, wherein the polyol contains 2 to 4 carbon atoms.

4. The dry eye composition of claim 1, wherein the polyol is a combination of glycerin and propylene glycol.

5. The dry eye composition of claim 1, having a viscosity that is a maximum of about 30 cps.

6. The dry eye composition of claim 1, wherein the average molecular weight of alginate is a minimum of about 50 kDa and a maximum of about 5000 kDa.

7. The composition of claim 1, wherein the concentration of alginate is a minimum of about 0.01 wt. % to about 5 wt. % based upon the total weight of the solution.

8. The composition of claim 1, wherein the ratio of guluronic acid monomer units to mannuronic acid monomer units is a minimum of about 25:75 and a maximum of less than 50:50.

9. The composition of claim 1, wherein the polyol is selected from the group consisting of glycerin, ethylene glycol, poly(ethylene glycol), propylene glycol, sorbitol, manitol and monosaccarides, disaccharides, neutral polysaccharides and oligosaccharides.

10. The composition of claim 1, wherein the polyol is selected from the group consisting of glycerin, ethylene glycol, propylene glycol, sorbitol, mannitol and monosaccharides.

11. The composition of claim 1, wherein the polyol is selected from the group comprising disaccharides, oligosaccharides and poly(ethylene glycol).

12. The composition of claim 1, wherein the alginate is characterized in that it has a Mark-Houwink number that is a minimum of about 0.6.

13. The composition of claim 1, wherein the composition contains low concentration of mono or divalent cations typically found in tear fluids.

14. The composition of claim 1, wherein the buffer(s) are selected from the group comprising phosphate buffer, borate buffer, MOPS buffer, citrate buffer, an aminoalcohol buffer and combinations thereof including but not limited to a phosphate/borate buffer and a citrate/borate buffer.

15. A method of treating dry eye comprising administering to an eye a composition comprising an aqueous solution of alginate and a polyol to the eye.

16. The method of claim 15, wherein the polyol contains 2 to 6 carbon atoms.

17. The method of claim 15, wherein the polyol contains 2 to 4 carbon atoms.

18. The method of claim 15, wherein the polyol is a combination of glycerin and propylene glycol.

19. The method of claim 15, wherein the composition has a viscosity that is a maximum of about 30 cps.

20. The method of claim 15, wherein the average molecular weight of alginate is a minimum of about 50 kDa and a maximum of about 5000 kDa.

21. The method of claim 15, wherein the concentration of alginate is a minimum of about 0.01 wt. % and a maximum of about 5 wt. % based upon the total weight of the composition.

22. The method of claim 15, wherein the alginate is characterized in that it has a Mark-Houwink number that is a minimum of about 0.6.

23. The method of claim 15, wherein the polyol is selected from the group consisting of glycerin, ethylene glycol, poly(ethylene glycol), propylene glycol, sorbitol, manitol and monosaccarides, disaccharides, neutral polysaccharides and oligosaccharides.

24. The method of claim 15, wherein the polyol is selected from the group consisting of glycerin, ethylene glycol, propylene glycol, sorbitol, mannitol and monosaccharides.

25. The method of claim 15, wherein the polyol is selected from the group comprising disaccharides, oligosaccharides and poly(ethylene glycol).

26. The method of claim 15, wherein the alginate is characterized in that it has a Mark-Houwink number that is a minimum of about 0.6 and a maximum of about 1.2.

27. The method of claim 15, wherein the composition contains low concentration of mono or divalent cations typically found in tear fluids.

28. The method of claim 15, wherein the buffer(s) are selected from the group comprising phosphate buffer, borate buffer, MOPS buffer, citrate buffer, an aminoalcohol buffer and combinations thereof including but not limited to a phosphate/borate buffer and a citrate/borate buffer.

29. The method of claim 15, wherein the pH of the composition is a minimum of about 4 and a maximum of about 8.

30. The method of claim 15, wherein the tonicity of the composition is a minimum of about 200 and a maximum of about 400.

31. A method of manufacturing a dry eye composition comprising combining in an aqueous solution ophthalmically pure alginate with ophthalmically pure polyol, wherein the ratio of the weight of alginate to weight of polyol in the composition is a minimum of 1:20 and a maximum of 20:1.

32. The method of claim 31, wherein the polyol contains 2 to 6 carbon atoms.

33. The method of claim 31, wherein the polyol contains 2 to 4 carbon atoms.

34. The method of claim 31, wherein the polyol is a combination of glycerin and propylene glycol.

35. The method of claim 31, wherein the composition has a viscosity that is a maximum of about 30 cps.

36. The method of claim 31, wherein the average molecular weight of alginate is a minimum of about 50 kDa and a maximum of about 5000 kDa.

37. The method of claim 31, wherein the concentration of alginate is a minimum of about 0.01 wt. % and a maximum of about 5 wt. % based upon the total weight of the composition.

38. The method of claim 31, wherein the alginate is characterized in that it has a Mark-Houwink number that is a minimum of about 0.6.

39. The method of claim 31, wherein the polyol is selected from the group consisting of glycerin, ethylene glycol, poly(ethylene glycol), propylene glycol, sorbitol, manitol and monosaccarides, disaccharides, neutral polysaccharides and oligosaccharides.

40. The method of claim 31, wherein the polyol is selected from the group consisting of glycerin, ethylene glycol, propylene glycol, sorbitol, mannitol and monosaccharides.

41. The method of claim 31, wherein the polyol is selected from the group comprising disaccharides, oligosaccharides and poly(ethylene glycol).

42. The method of claim 31, wherein the alginate is characterized in that it has a Mark-Houwink number that is a minimum of about 0.6 and a maximum of about 1.2.

43. The method of claim 31, wherein the composition contains low concentration of mono or divalent cations typically found in tear fluids.

44. The method of claim 31, wherein the buffer(s) are selected from the group comprising phosphate buffer, borate buffer, MOPS buffer, citrate buffer, an aminoalcohol buffer and combinations thereof including but not limited to a phosphate/borate buffer and a citrate/borate buffer.

45. The method of claim 31, wherein the pH of the composition is a minimum of about 4 and a maximum of about 8.

46. The method of claim 31, wherein the tonicity of the composition is a minimum of about 200 mOsm/kg and a maximum of about 400 mOsm/kg.

47. A dry eye composition comprising an aqueous solution of alginate having a molecular weight that is a minimum of about 50 kDa and a maximum of about 5000 kDa and a polyol wherein the composition does not contain a active pharmaceutical agent.

48. The dry eye composition of claim 47, wherein the polyol contains 2 to 6 carbon atoms.

49. The dry eye composition of claim 47, wherein the polyol contains 2 to 4 carbon atoms.

50. The dry eye composition of claim 47, wherein the polyol is a combination of glycerin and propylene glycol.

51. The dry eye composition of claim 47, having a viscosity that is a maximum of about 30 cps.

52. The dry eye composition of claim 47, wherein the average molecular weight of alginate is a minimum of about 50 kDa and a maximum of about 5000 kDa.

53. The composition of claim 47, wherein the concentration of alginate is a minimum of about 0.01 wt. % and a maximum of about 5 wt. % based upon the total weight of the composition.

54. The composition of claim 47, wherein the alginate is characterized in that it has a Mark-Houwink number that is a minimum of about 0.6.

55. The composition of claim 47, wherein the polyol is selected from the group consisting of glycerin, ethylene glycol, poly(ethylene glycol), propylene glycol, sorbitol, manitol and monosaccarides, disaccharides, neutral polysaccharides and oligosaccharides.

56. The composition of claim 47, wherein the polyol is selected from the group consisting of glycerin, ethylene glycol, propylene glycol, sorbitol, mannitol and monosaccharides.

57. The composition of claim 47, wherein the polyol is selected from the group comprising disaccharides, oligosaccharides and poly(ethylene glycol).

58. The composition of claim 47, wherein the alginate is characterized in that it has a Mark-Houwink number that is a minimum of about 0.6 and a maximum of about 1.2.

59. The composition of claim 47, wherein the composition contains low concentration of mono or divalent cations typically found in tear fluids.

60. The composition of claim 47, wherein the buffer(s) are selected from the group comprising phosphate buffer, borate buffer, MOPS buffer, citrate buffer, an aminoalcohol buffer and combinations thereof including but not limited to a phosphate/borate buffer and a citrate/borate buffer.

61. A method of manufacturing a dry eye composition comprising combining in an aqueous solution ophthalmically pure alginate having a molecular weight that is a minimum of about 50 kDa and a maximum of about 5000 kDa with ophthalmically pure polyol.

62. The method of claim 61, wherein the ratio of the weight of alginate to weight of polyol in the composition is a minimum of 1:4 and a maximum of 4:1.

63. The method of claim 61, wherein the polyol contains 2 to 6 carbon atoms.

64. The method of claim 61, wherein the polyol contains 2 to 4 carbon atoms.

65. The method of claim 61, wherein the polyol is a combination of glycerin and propylene glycol.

66. The method of claim 61, wherein the composition has a viscosity that is a maximum of about 30 cps.

67. The method of claim 61, wherein the average molecular weight of alginate is a minimum of about 50 kDa and a maximum of about 5000 kDa.

68. The method of claim 61, wherein the concentration of alginate is a minimum of about 0.01 wt. % and a maximum of about 2.0 wt. % based upon the total weight of the composition.

69. The method of claim 61, wherein the alginate is characterized in that it has a Mark-Houwink number that is a minimum of about 0.6.

70. The method of claim 61, wherein the polyol is selected from the group consisting of glycerin, ethylene glycol, poly(ethylene glycol), propylene glycol, sorbitol, manitol and monosaccarides, disaccharides, neutral polysaccharides and oligosaccharides.

71. The method of claim 61, wherein the polyol is selected from the group consisting of glycerin, ethylene glycol, propylene glycol, sorbitol, mannitol and monosaccharides.

72. The method of claim 61, wherein the polyol is selected from the group comprising disaccharides, oligosaccharides and poly(ethylene glycol).

73. The method of claim 61, wherein the alginate is characterized in that it has a Mark-Houwink number that is a minimum of about 0.6 and a maximum of about 1.2.

74. The method of claim 61, wherein the composition contains low concentration of mono or divalent cations typically found in tear fluids.

75. The method of claim 61, wherein the buffer(s) are selected from the group comprising phosphate buffer, borate buffer, MOPS buffer, citrate buffer, an aminoalcohol buffer and combinations thereof including but not limited to a phosphate/borate buffer and a citrate/borate buffer.

76. The method of claim 61, wherein the pH of the composition is a minimum of about 6.0 and a maximum of about 8.0.

77. The method of claim 61, wherein the tonicity of the composition is a minimum of about 240 mOsmo/l and a maximum of about 320 mOsmo/l.
